(19) United States
(12) Patent Application Publication
(10) Pub. No.: US 2026/0306249 A1
(43) Pub. Date: Oct. 8, 2026

(54) SELF-ADJUSTING SUSPENSION SYSTEMS FOR INTRAOCULAR LENSES

(71) Applicant: OCUMETICS TECHNOLOGY CORP., Calgary (CA)

(72) Inventors: Garth T. WEBB, Langley (CA); Adriana Torres FLORES, Monterrey (MX); Jorge Alberto Gutierrez VALENZUELA, Hermosillo (MX)

(21) Appl. No.: 19/481,514

(22) PCT Filed: May 3, 2024
(86) PCT No.: PCT/CA2024/050611

§ 371 Date: Nov. 4, 2025

(30) Foreign Application Priority Data
May 4, 2023 (CA) 3198760

(51) Int. Cl. A61F 2/16

(52) U.S. Cl. CPC A61F 2/16; A61F 2002/169; A61F 2002/16901; A61F 2250/001

(57) ABSTRACT

In some embodiments the invention relates to a self-adjusting suspension system positionable within an ocular capsular cavity comprising a first ring configured for holding an intraocular lens; an expandable second ring at least partially surrounding the first ring; and connectors for coupling the second ring to the first ring. The system is adjustable between a compressed configuration for deployment into the capsular cavity, for example during a surgical installation procedure, and an expanded configuration for secure engagement with an inner wall of the capsular cavity. In some embodiments the system may comprise a plurality of biasing elements for biasing the system from the compressed position to the expanded position, wherein at least some of the biasing elements apply a radial force to first portions of the second ring and at least some other biasing elements apply a tangential force to second portions of the second ring. Once deployed, the second ring dynamically supports the first ring within the capsular cavity such that the lens is aligned with a preferred visual axis of a recipient eye. The invention also comprises methods of deploying self-adjusting suspension systems and uses thereof for supporting a lens within an ocular capsular bag cavity.

### REFERENCE TO RELATED APPLICATIONS

The present application claims priority to Canadian patent application no. 3198760 filed 4 May 2023 entitled SELF-ADJUSTING SUSPENSION SYSTEMS FOR INTRAOCULAR LENSES. The present invention relates to subject matter described in the applicant's U.S. patent application Ser. No. 12/671,573 entitled INFLATABLE INTRAOCULAR LENS/LENS RETAINER filed 12 Aug. 2008; U.S. provisional application no. 61/761,569 filed 6 Feb. 2013 entitled LASER SCULPTED COMPARTMENTS WITHIN SUSPENSION SYSTEMS FOR INTRAOCULAR LENSES; international application publication no. WO 2014/121391 A1 published 14 Aug. 2014 entitled EXPANDABLE SUSPENSION SYSTEMS FOR INTRAOCULAR LENSES; and international application publication no. WO 2017/181295 A1 published 26 Oct. 2017 entitled COLLAPSIBLE CAVITIES WITHIN SUSPENSION SYSTEMS FOR INTRAOCULAR LENSES. All of the above applications and publications are incorporated herein by reference.

### TECHNICAL FIELD

The present invention relates to suspension systems for intraocular lenses, methods of intraocular deployment thereof, and uses thereof.

### BACKGROUND

Intraocular lens designs have evolved over the past decades from passive solid-state lenses to dynamic accommodating optical systems. This evolution has been propelled by a demand for improved safety and efficacy within the field of cataract and refractive surgery, to minimize, and ultimately eliminate dependency upon extraneous visual aids, such as eye glasses, contact lenses and the like.

By their nature, accommodating intraocular lenses require complex suspension systems and often bulky optical apparatus to re-establish the natural bio-kinetics of the muscles and ligaments within the eye. By contrast, solid-state intraocular lenses and their suspension systems tend to be streamlined in an effort to minimize intraoperative trauma. Several clinically serious shortcomings are associated with prior art lens suspension systems, including dislodged lens haptics, unstable lens centration, cystoid macular oedema, vitreous detachment and retinal detachment.

Some prior art suspension systems comprise laterally projecting arms or other appendages. Such systems require precise surgical placement or other manipulation in vivo to avoid possible dislodgement, ocular irritation or other potentially serious side effects.

There is need for improved suspension systems for both solid-state and accommodating intraocular lenses. In particular, there is a need for improved systems, methods and uses for reducing risk factors associated with lens replacement surgery by achieving a simplified installation capacity and improved efficacy in vivo.

The forgoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described have been reduced or eliminated, while other embodiments are directed toward other improvements.

One aspect of the invention relates to a lens suspension system positionable within an ocular capsular cavity comprising a first ring configured for holding a lens; an expandable second ring at least partially surrounding the first ring; and connectors for coupling the second ring to the first ring, wherein the system is adjustable between a compressed configuration for deployment into the capsular cavity and an expanded configuration for engagement with an inner wall of the capsular cavity. In some embodiments the second ring comprises at least one biasing element for biasing the second ring toward the expanded configuration. In some embodiments the second ring comprises a plurality of spaced-apart second segments and a plurality of hinges, wherein each of the hinges is positionable between an adjacent pair of the second segments. In some embodiments at least some of the hinges are configured to apply opposing tangential forces to the adjacent pair of second segments when the system is adjusted between the compressed and expanded configurations. In some embodiments the connectors may be configured to apply radial forces to the second ring when the system is adjusted between the compressed and expanded configurations to symmetrically alter the spacing between the first and second rings. In some embodiments the system may comprise a plurality of biasing elements for biasing the system from the compressed position to the expanded position, wherein at least some of the biasing elements apply a radial force to first portions of the second ring and at least some other biasing elements apply a tangential force to second portions of the second ring.

In another aspect, the invention relates to a method of deploying an intraocular lens in an ocular capsular bag cavity having an internal wall, the method comprising providing a lens suspension system comprising an first ring configured for holding the lens; an expandable second ring at least partially surrounding the first ring; and connectors for coupling the second ring to the first ring; and introducing the lens suspension system into the cavity in a compressed configuration and allowing the system to adjust from the compressed configuration to an expanded configuration wherein outer surfaces of the second ring securely engage the internal wall of the cavity. In some embodiments the second ring and the first ring may be introduced into the cavity in separate steps and then connected together therein.

In another aspect, the invention relates to the use of a suspension system as described herein for supporting a lens within an ocular capsular bag cavity. In some embodiments the use enables dynamic adjustment of the suspension system in vivo in response to changes in the size of the capsular bag cavity and/or forces applied thereto.

In addition to the exemplary aspects and embodiments described above, further aspects and embodiments will become apparent by reference to drawings and a study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a cross sectional schematic representation of the human eye.

FIG. 2 is a cross sectional schematic representation of the human eye of FIG. 1 showing an embodiment of the self-adjusting suspension system of the invention positioned within its lens capsular bag cavity.

FIG. 3A is a perspective view of an embodiment of the suspension system of the invention.

FIG. 3B is a perspective view of an alternative embodiment of the suspension system.

FIG. 3C is a perspective view of a further alternate embodiment of the suspension system having a non-continuous first ring.

FIG. 4 is a top view of suspension system of FIG. 3A.

FIG. 5A is a first enlargement of area A shown in FIG. 4 and showing a hinge in a compressed configuration.

FIG. 5B is a second enlargement of area A shown in FIG. 4 and showing a hinge in an expanded configuration.

FIG. 6A is an enlargement of area B shown in FIG. 4 and showing a spoke connector in a compressed configuration.

FIG. 6B is a second enlargement of area B shown in FIG. 4 and showing a spoke connector in an expanded configuration.

FIG. 7 is a top view of an embodiment of the suspension system.

FIG. 8A is a first cross sectional view taken along lines C-C of FIG. 7 showing two radial spoke connectors of the suspension system in their compressed configuration.

FIG. 8B is a second cross sectional view taken along lines C-C of FIG. 7 showing two radial spokes of the suspension system in an expanded configuration.

FIG. 8C is a second cross sectional view taken along lines C-C of FIG. 7 showing two radial spokes of the suspension system in an expanded configuration and denoting the definition of a V-shaped spring.

FIG. 9 is a top view of a further embodiment of the suspension system.

FIG. 10 is a top view of a further embodiment of the suspension system.

FIG. 11A is a top view of a further embodiment of the suspension system.

FIG. 11B is a top view of a further embodiment of the suspension system.

### DESCRIPTION

Throughout the following description, specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive sense.

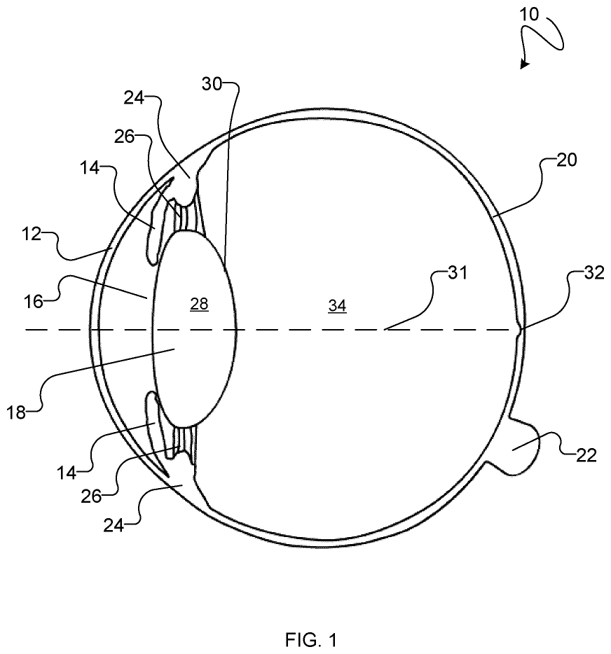

FIG. 1 illustrates a schematic cross-section of a human eye 10. Eye 10 includes a cornea 12, iris 14, pupillary aperture 16, lens 18, retina 20, and optic nerve 22. Eye 10 has the capacity to adjust its focus from distant objects to near objects due to the ability of lens 18 to change shape, a phenomenon known as accommodation. Lens 18 can change shape by virtue of the ciliary muscle 24 acting upon zonules 26 comprising suspensory ligaments. When ciliary muscle 24 contracts, relaxation of zonular tension occurs which causes lens 18 to assume a more spherical shape, resulting in increased dioptic power which helps bring nearer objects into focus. Relaxation of ciliary muscle 24 causes zonular tension to increase resulting in a flattening of lens 18 which helps bring more distant objects into focus.

Lens 18 is located within a lens capsule or capsular bag 28 having an inner surface or wall 30. The equatorial perimeter of the inner surface or wall 30 of capsular bag 28 may comprise an anatomical groove sometimes referred to as the sulcus. Cataract surgery and other ocular procedures can involve the removal of a natural lens 18 from the capsular bag 28 and the introduction of a synthetic lens 18A within the vacant cavity of capsular bag 28, for example in engagement with the sulcus. The synthetic lens 18A may be a solid-state or accommodating intracocular lens, and is typically supported within the cavity of capsular bag 28 by a suspension system.

With reference to FIG. 1, the visual axis 31 of eye 10 is an imaginary line (shown in dotted outline in the drawings) that connects an object in space with the respective foveola 32 of retina 20. Light emanating from an object in space travels through transparent structures of eye 10 and carries on through pupillary aperture 16, lens capsular bag 28, natural lens 18, and vitreous chamber 34 to eventually focus upon foveola 32.

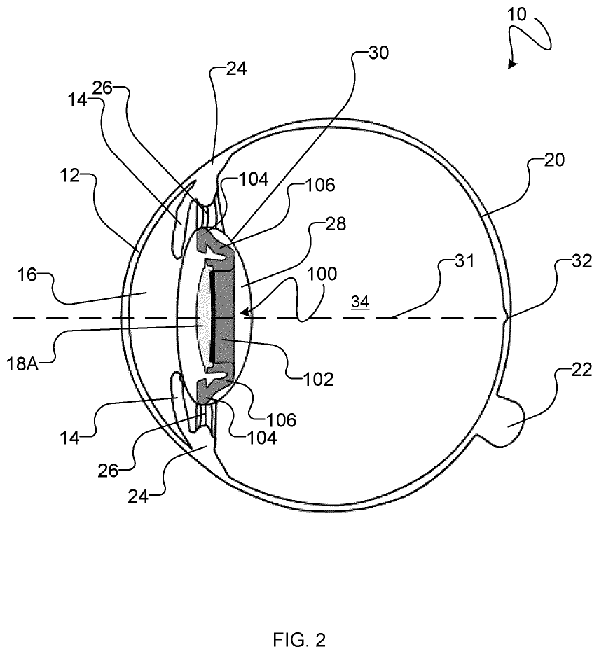

The present invention is directed to self-adjusting suspension systems, methods of intraocular deployment thereof, and uses thereof adapted for positioning a synthetic lens 18A within the vacant cavity of a lens capsular bag 28 (i.e. after removal of a natural lens 18). FIG. 2 illustrates an embodiment of a suspension system 100 deployed within a capsular bag 28 wherein an outer portion of system 100 is in engagement with an equatorial perimeter region or sulcus of inner wall 30. As described herein, in some embodiments suspension system 100 may comprise a first or inner ring 102 for supporting synthetic lens 18A and a second or outer ring 104 which at least partially surrounds first ring 102 and is coupled thereto by a plurality of connectors 106. As described herein, suspension system 100 is adjustable between a compressed configuration for introducing system 100 into the cavity of a lens capsular bag 28 and an expanded, deployed configuration wherein outer portions of second ring 104 securely engage inner wall 30 of capsular bag 28. In the deployed configuration second ring 104 and connectors 106 maintain first ring 102 within capsular bag 28 in an optimal intraocular orientation such that synthetic lens 18A is in alignment with a preferred visual axis 31 of a recipient eye 10.

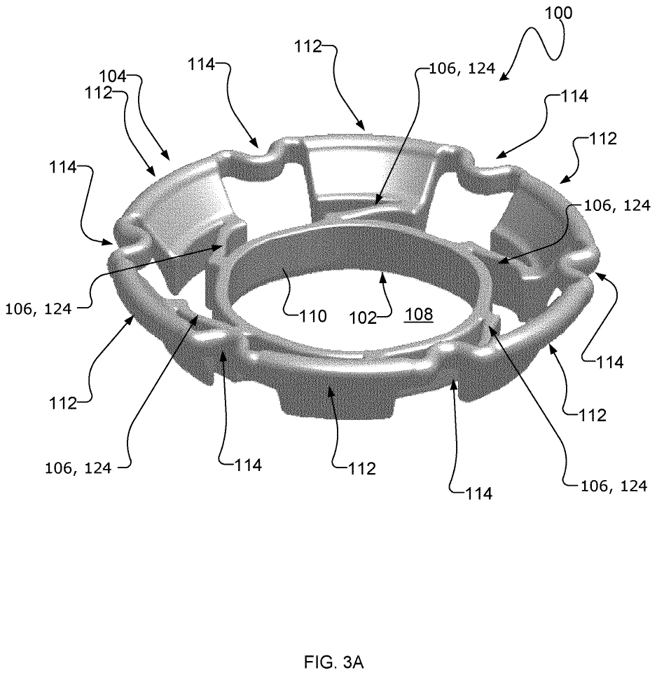

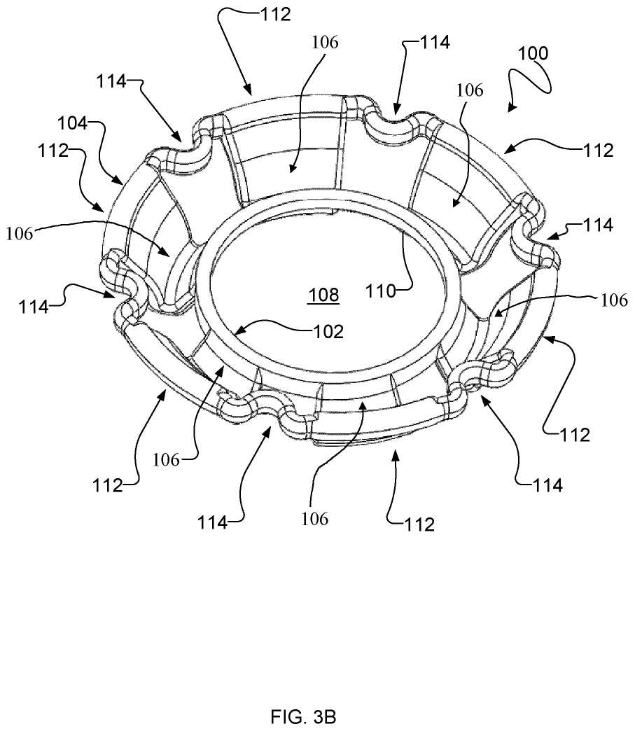

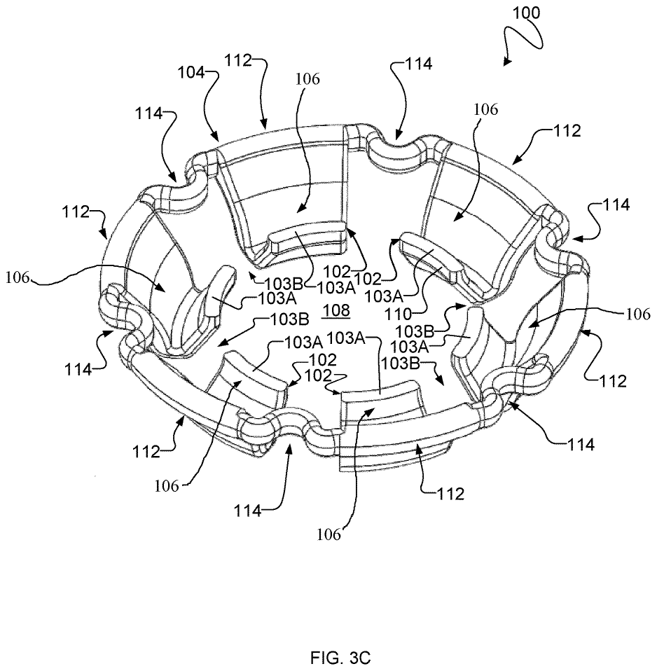

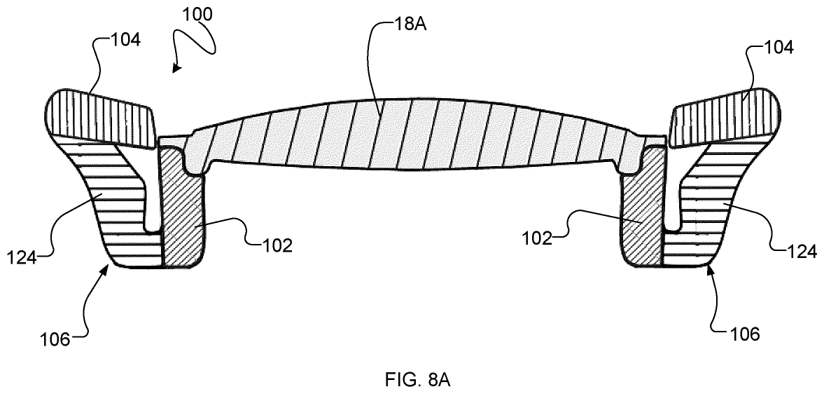

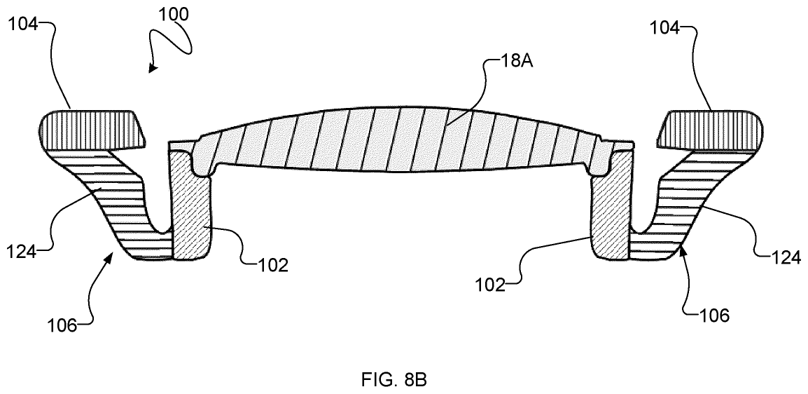

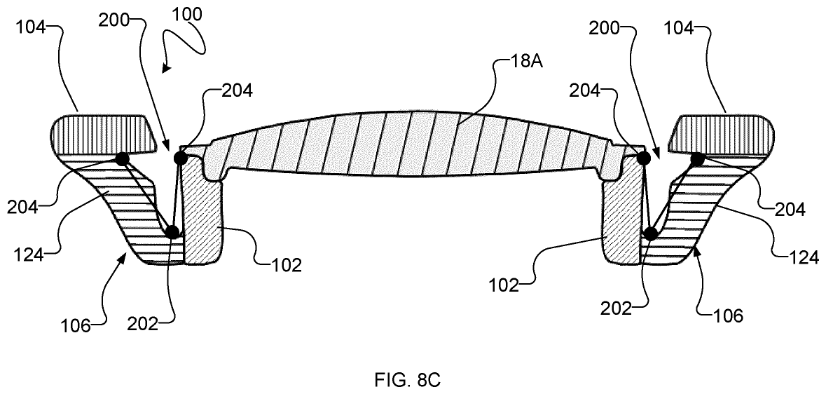

FIGS. 3A-3C are perspective views showing different embodiments of suspension system 100 in an expanded, low energy habitual configuration. In some embodiments, suspension system 100 is resiliently deformable to enable adjustment thereof between compressed and expanded configurations, for example during surgical positioning of system 100 within capsular bag 28 and/or after intraocular deployment of system 100 within capsular bag 28 after second ring 104 is seated in secure engagement with inner wall 30. After intraocular deployment, deformation of system 100 may be due to the ciliary muscle contraction or relaxation and corresponding changes in zonular tension acting upon system 100. By way of example, FIG. 8A is a cross sectional view of an embodiment of system 100 in a compressed, high energy configuration and FIG. 8B is a cross sectional view of the same embodiment in an expanded, low energy or habitual configuration.

As explained above, when system 100 is initially deployed in vivo within capsular bag 28 system 100 ordinarily self-adjusts from a compressed, high energy state to an expanded, lower energy state. However, in some embodiments system 100 does not adjust to a fully expanded, lowest energy state in vivo. Rather, when deployed within capsular bag 28, the expansionary forces described herein bias outer surfaces of second ring 104 outwardly into secure engagement with capsular bag inner wall 30. Such secure engagement prevents undesired movement or dislodgement of system 100 and accompanying synthetic lens 18A in vivo and enables system 100 to dynamically self-adapt to changing in vivo conditions. For example, system 100 may be surgically deployed in a child having a relatively small capsular bag 28. As the child grows the capsular bag 28 becomes correspondingly larger in size and system 100 can further expand outwardly to remain in secure engagement with inner wall 30. Thus system 100 can compensate for growth-related changes in the dimensions of capsular bag 28. Conversely, if capsular bag 28 shrinks in size due to fibrotic changes, age-related atrophy or other like, system 100 can adjust in size accordingly without applying undue forces to inner wall 100.

Relatedly, deployment of system 100 in vivo helps maintain a significant separation between the anterior aspect and posterior aspect of the capsular bag wall 30 since second ring 104 is pushing outwardly generally symmetrically, for example against the sulcus of the lens capsule. This prevents such anterior and posterior wall portions from coming into significant contact and knitting together due to fibrosis (as is typical in some conventional lens suspension systems). Further, in some embodiments second ring 104 and/or connectors 106 are sufficiently thick that they act as a physical barrier to further ensure that there is little potential for anterior and posterior wall portions of the lens capsule to come into significant contact and adhere to one another.

As discussed above, system 100 can also dynamically adjust to external forces applied to capsular bag 28, for example caused by natural contraction and relaxation of ciliary muscle 24. In some embodiments such external forces can cause changes in size and/or shape of synthetic lens 18A to enable ocular accommodation. As the size of second ring 104 dynamically self-adjusts to changes in the size of capsular bag 28 and/or forces applied thereto, first ring 102 serves as a support structure that holds a synthetic optical lens 18A (shown in FIG. 2) in alignment with visual axis 31 to facilitate the eye's ability to focus light upon the retina 20 in the absence of natural lens 18. Synthetic optical lens 18A can exist as a separate element that lays upon the rim of first ring 102 or it can be molded directly onto first ring 102 as a single unit. First ring 102 comprises a transparent or hollow central aperture 108. The wall or walls 110 of first ring 102 lining aperture 108 are preferably constructed with opaque or non-reflective materials to limit interference caused by stray light.

In the embodiments illustrated in perspective view in FIGS. 3A and 3B, first ring 102 is a continuous, generally annular or oval-shaped structure supporting lens 18. In other embodiments first ring 102 may be any other size, shape, profile and/or pattern suitable for supporting lens 18A. As shown in FIG. 3C, in some alternative embodiments first ring 102 may comprise a plurality of spaced-apart first segments 103A having gaps 103B therebetween. Such first segments 103A may be connected together either directly or indirectly by means of connectors 106 coupled to second ring 104 at least partially surrounding first ring 102. In some embodiments the first segments 103A may comprise legs secured to the lens, for example by an adhesive, and then coupled to second ring 104.

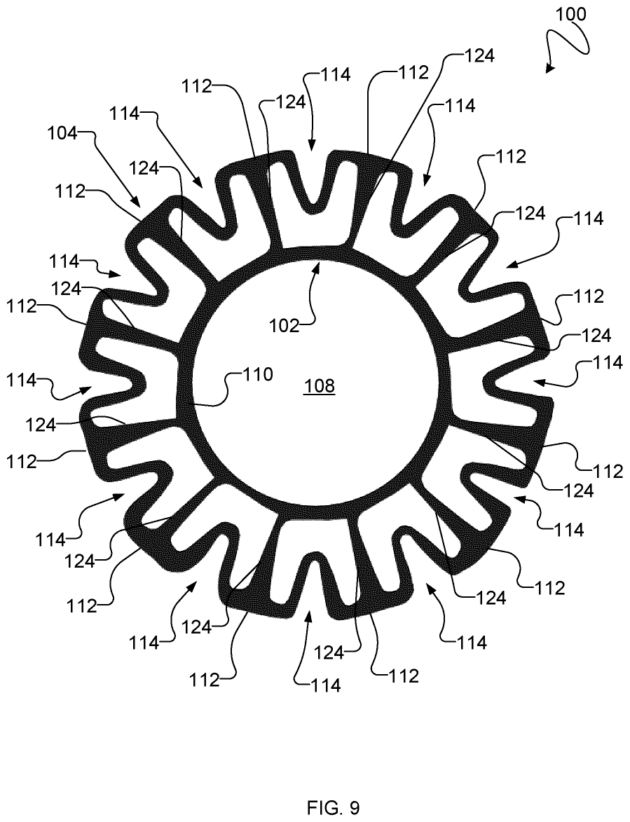

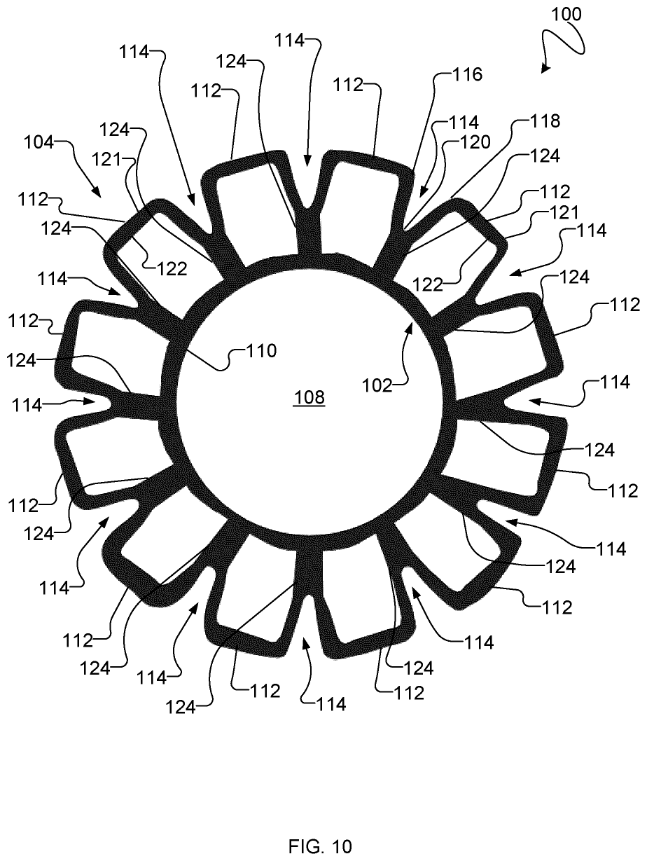

In some embodiments second ring 104 comprises a plurality of second segments 112 and a plurality of hinges 114. In some of the embodiments each of the hinges 114 is positionable between an adjacent pair of second segments 112 to couple segments 112 together. For example, as shown in FIGS. 3A-3C, 4, 7, 9 and 10, second ring 104 may be configured as a 360° continuum of alternating second segments 112 and hinges 114. Each second segment 112 may comprise an outer surface 121 and an inner surface 122. In the illustrated embodiments the outer circumference of second ring 104 defined by second segments 112 is shown as having a generally circular profile. However, the perimeter profile or other features of second ring 104 can be fashioned to suit any desired shape, size, orientation and/or pattern. Further, the number of second segments 112 and hinges 114 may vary in different embodiments without departing from the invention. For example, in some illustrated embodiments (e.g. FIGS. 3A-3C, 4 and 7) second ring 104 comprises six second segments 112 and six hinges 114; in other illustrated embodiments (e.g. FIGS. 9 and 10) second ring 104 comprises twelve second segments 112 and 12 hinges 114.

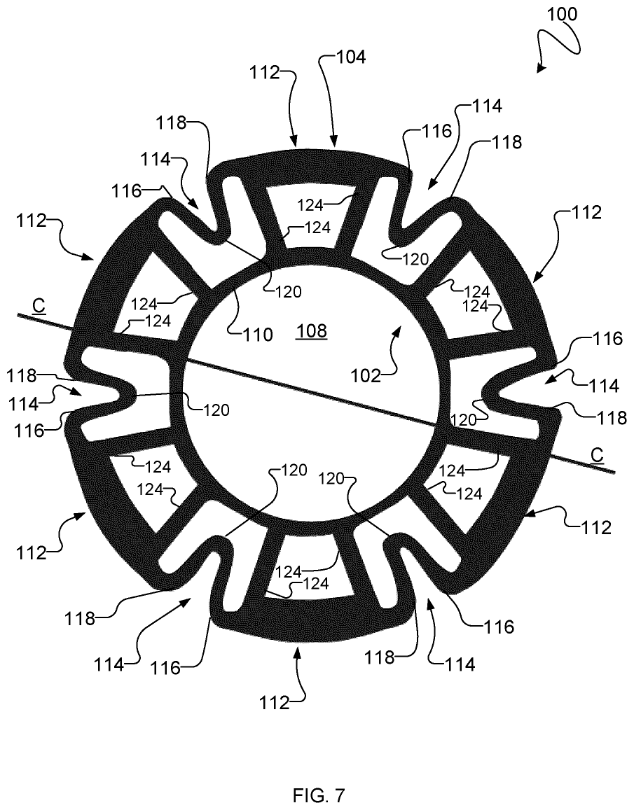

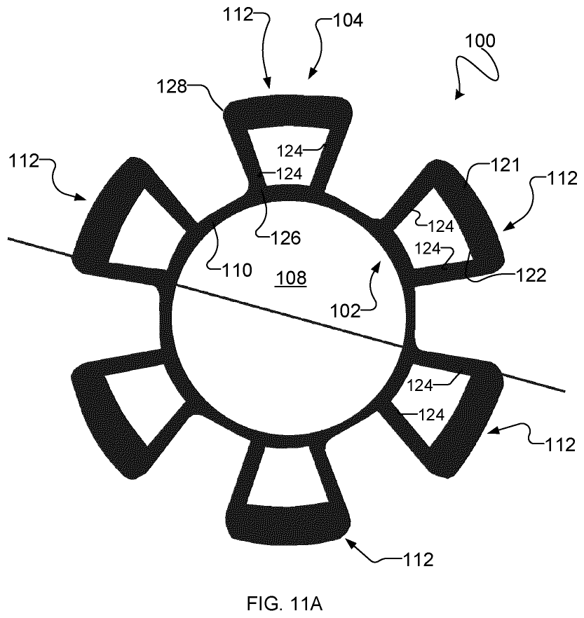

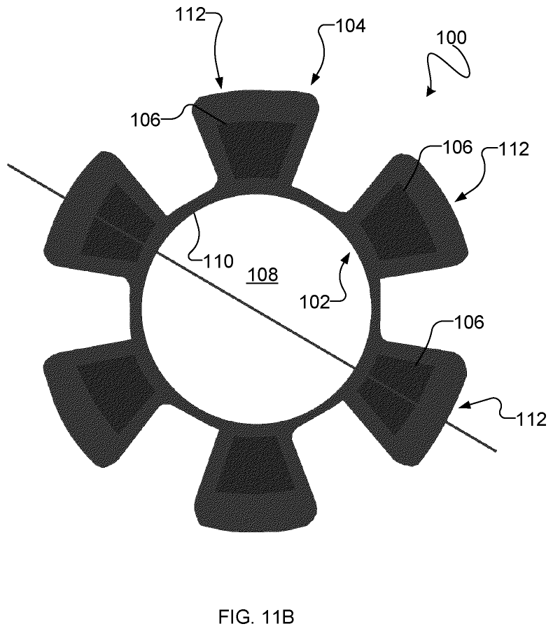

In some embodiments hinges 114 may be omitted entirely, as illustrated for example in FIG. 11A and11B. In such example embodiments the outer circumference of second ring 104 is defined by a plurality of disconnected second segments 112. Accordingly, second ring 104 is interrupted rather than continuous. In such example embodiments each second segment 112 is connected to first ring 102 by one or more connectors 106 which extend from first ring 102 to second ring 104. In the example embodiment of FIG. 11A connectors 106 comprise a pair of spaced-apart spokes 124 similar to the embodiment of FIG. 7. In the example embodiment of FIG. 11B each connector 106 is a single member similar to the embodiments of FIGS. 3B and 3C.

In some possible embodiments the length of some or all second segments 112 coupled by hinges 114 can be relatively short (FIG. 9). In other embodiments second segments 112 may be very small or omitted entirely and hinges 114 may be coupled directly together. That is, in some embodiments second ring 104 may comprise a plurality of hinges 114 connected end-to-end around a 360° continuum of second ring 104 without the need for any identifiable segments 112. In such embodiments outer portions of hinges 114, for example contact points located an the juncture between adjacent hinges 114, define an outer surface of second ring 104 for engaging inner wall 30 of capsular bag 28 when suspension system 100 is deployed therein.

Accordingly, in some embodiments outer surface 121 of each of the second segments 112 may engage inner wall 30 of capsular bag 28 in vivo, e.g. at least partially at the equatorial sulcus. In other embodiments, some of the segments 112 may be inwardly recessed to not contact inner wall 30, i.e. to purposely avoid engagement with the sulcus or other surfaces of inner wall 30 in some circumstances.

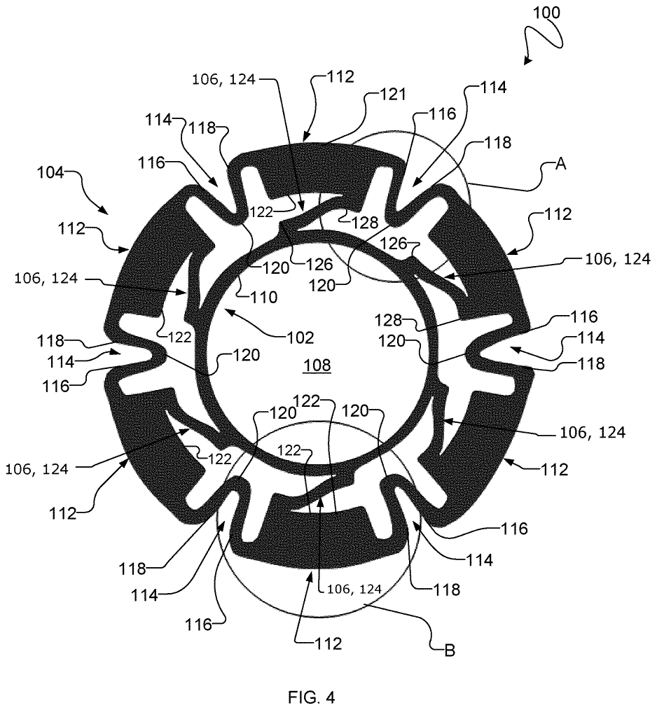

In some embodiments hinges 114 may function as biasing elements to bias suspension system 100 from a compressed, high energy configuration to an expanded, lower energy configuration. More particularly, hinges 114 may be resilient deformable to apply generally opposing tangential forces to respective pairs of second segments 112 when suspension system 100 is adjusted from a compressed configuration to the expanded configuration. In some embodiments each hinge 114 is generally V-shaped and comprises a first portion 116 connected to a first one of the adjacent pair of second segments 112 and a second portion 118 connected to a second one of the adjacent pair of second segments 112. Inner ends of the first and second portions 116, 118 are connected together at a joinder 120 disposed between the first one and the second one of the adjacent pair of second segments 112 (e, g, FIG. 4).

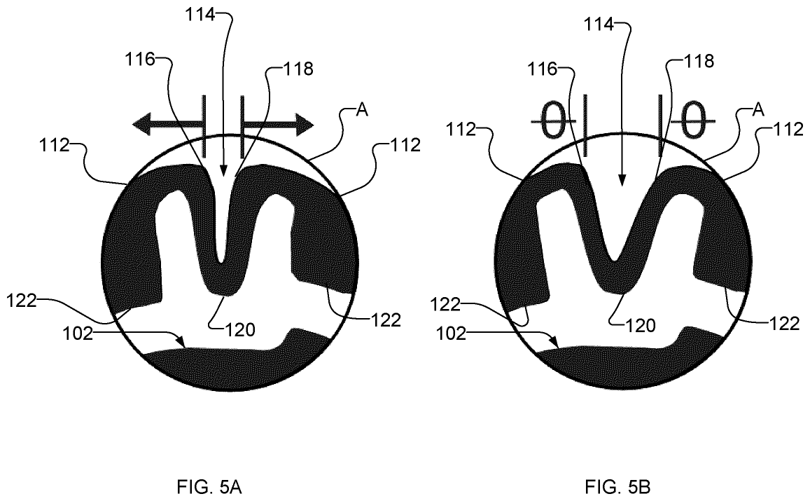

FIGS. 5A and 5B are enlarged views of Area A shown in FIG. 4 showing a pair of second segments 112 connected by a hinge 114. FIG. 5A shows system 100 in a compressed, high energy configuration having a relatively small spacing between first and second portions 116, 118 of hinge 114. FIG. 5B shows system 100 in an expanded, low energy configuration having a larger spacing between first and second portions 116, 118 of hinge 114. Since in some embodiments hinge 114 is resiliently deformable as discussed above, the release of the compression of first and second portions 116, 118 toward one another results in the application of opposing tangential forces to second segments 112 in the direction of the arrows shown in FIG. 5A, i.e. in opposing directions generally perpendicular to a radial axis of second ring 104. The release of compression of the plurality of hinges 114 causes expansion of second ring 104, enabling secure engagement of suspension system 100 with inner wall 30 of capsular bag 28. Suspension system 100 is thus self-adjusting to ensure secure deployment within capsular bag cavities of differing sizes, i.e. within an acceptable anatomical range of sizes. In some embodiments an increase in the number of hinges 114 enhances the range of expansion of second ring 104 and therefore its adaptability to a greater range of lens capsular bag 28 sizes. Although FIG. 5B shows system 100 in a low energy configuration with tangential force vectors at 0, it will be understood that in vivo system 100 may be partially compressed to securely engage wall 30, i.e. system 100 may be operable in a partially energized state with force vectors greater than 0. That is, in vivo system 100 may change between differing states of compression and expansion to dynamically adjust to external forces applied to capsular bag 28, for example caused by natural contraction and relaxation of ciliary muscle 24 as discussed above.

In some embodiments, second ring 104 may be configured such that at least some second segments 112 have a relatively narrow profile, i.e. having a relatively small width between outer surface 121 thereof and an inner surface 122 thereof (e.g. FIG. 10). In other embodiments at least some second segments 112 having a relatively thick profile, i.e. having a relatively large width between surfaces 121, 122 (e.g. FIG. 4). In such embodiments inner surface 122 of second segments 112 may be disposed closely spaced-apart from or in contact with wall 110 of first ring 102. As will be apparent to a person skilled in the art, the size, shape, profile and/or pattern of second segments 112 can vary in different embodiments without departing from the invention.

Unlike some prior devices, in some embodiments suspension system 100 does not comprise any arms or other appendages projecting outwardly from second ring 104. Rather, as discussed above, second ring 104 may define a uniform outer surface, such as an annular 360° continuum of alternating second segments 112 and hinges 114. Thus in some embodiments system 100 comprising second ring 104 does not need to be deployed in any particular orientation or require post-deployment adjustment or precise seating of laterally extending arms or the like.

As discussed above, first ring 102 is coupled to second ring 104 by a plurality of connectors 106. Connectors 106 may be configured to hold first ring 102 centrally in a coaxial orientation relative to second ring 104. Further, at least some connectors 106 may be configured to bias suspension system 100 from a compressed configuration to an expanded configuration. For example, as described herein, connectors 106 may apply radial forces to second ring 104 to increase the separation between first and second rings 102, 104 when system 100 is adjusted from a compressed to an expanded configuration.

With reference to FIGS. 4, 7, 9 and 10 in some embodiments connectors 106 comprise a plurality of spaced-apart spokes 124 which each extend from first ring 102 to inner surface 122 of a corresponding second segment 112 of second ring 104. In some embodiments a single spoke 124 may be connected to an end portion of a corresponding second segment 112 (FIG. 4). In other embodiments multiple spokes 124 may be connected to a corresponding second segment 112, for example a pair of spokes 124 may be connected to opposed ends of a corresponding second segment 112 (FIG. 7). In other embodiments one or more spokes 124 may be connected to a central portion of a corresponding second segment 112 (FIG. 9). In still other embodiments each spoke 124 may be connected to a hinge 114 of second ring 104 rather than a second segment 112 thereof. For example, at least some spokes 124 may be connected to a hinge joinder 120 disposed between an adjacent pair of second segments 112 (FIG. 10).

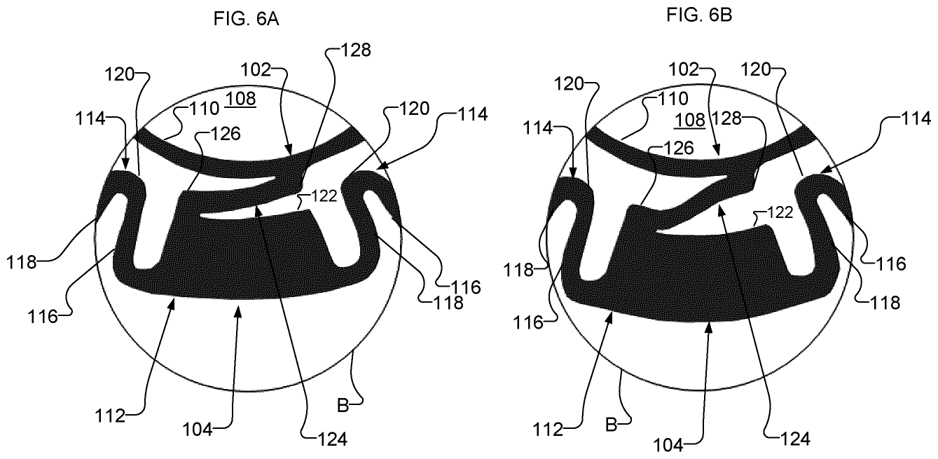

In some embodiments each spoke 124 extends between an inner end 126 thereof and an outer end 128 thereof in a direction intersecting a radial axis of inner ring 102. For example, in the compressed configuration illustrated in of FIG. 6A, spoke 124 extends in a direction generally tangential to the curvature of first ring 102. In other embodiments one or more spokes 124 may extend generally radially between first ring 102 and second ring 104 (FIGS. 7, 9 and 10), or part-way between a radial orientation and a tangential orientation. As will be apparent to a person skilled in the art, the size, shape, profile, orientation and/or pattern of connectors 106, such as spokes 124, can vary in different embodiments without departing from the invention.

As indicated above, connectors 106, such as spokes 124, may function as resiliently deformable elements biasing suspension system 100 from a compressed configuration to an expanded configuration. For example, when system 100 is in a compressed configuration suitable for surgical deployment into capsular bag 28, second (outer) ring 104 may be compressed toward or in contact with first (inner) ring 102 to minimize the overall size of system 100. In such a compressed configuration outer ends 128 of spokes 124 may be compressed toward first ring 102 to reduce the distance therebetween (FIG. 6A). When the aforesaid compression is released, spokes 124 may return to an uncompressed state, thereby biasing second ring 104 outwardly in a radial direction away from first ring 102 to the expanded configuration (FIG. 6B).

Accordingly, in some embodiments suspension system 100 may comprise one biasing element, such as at least some hinges 114 or at least some spokes 124 for biasing system 100 from a compressed, high energy configuration to an expanded, lower energy configuration. In other embodiments suspension system 100 may comprise a plurality of biasing elements, such as a combination of at least some hinges 114 and at least some spokes 124. As described above, the different biasing elements may apply differing expansionary forces to second ring 104, such as a combination of both tangential forces and radial forces for adjusting second ring 104 to an expanded, securely deployed position within capsular bags 28 of different sizes. As will be apparent to a person skilled in the art, many other biasing elements or combinations of biasing elements may be envisioned for enabling adjustment of suspension system 100 between a compressed configuration for deployment into a capsular bag 28 and an expanded configuration for engagement with an inner wall 30 of capsular bag 28.

With reference to the embodiments of FIGS. 11A and 11B, hinges 114 are omitted. As described above, connectors 106, such as spokes 124, may function as resiliently deformable elements biasing suspension system 100 from a compressed configuration to an expanded configuration. For example, when system 100 is in a compressed configuration suitable for surgical deployment into capsular bag 28, second ring 104 may be compressed toward or in contact with first ring 102 to minimize the overall size of system 100. In such a compressed configuration outer ends 128 of spokes 124 may be compressed toward first ring 102 to reduce the distance therebetween. When the aforesaid compression is released, spokes 124 may return to an uncompressed state, thereby biasing second ring 104 comprising the plurality of disconnected second segments 112 outwardly in a radial direction away from first ring 102 to the expanded configuration, causing the outer wall 121 of each segment 112 to press against inner wall 30 of capsular bag 28. The connectors 106 of FIG. 11B would function similarly. Owing to the structural strength and elasticity of the component parts of system 100, including first ring 102, second ring 104 and connectors 106, system 100 may be reliably adjusted between a compressed, high energy configuration and an expanded, lower energy configuration without the need for hinges 114. In some embodiments first ring 102 may serve as a ballast that stores kinetic energy in the compressed, high energy configuration and releases it when system 100 is adjusted to the expanded, lower energy configuration, such as when system 100 is deployed within a capsular bag 28. In the embodiments of FIGS. 11A and 11B the biasing forces are sufficient to deploy system 100 within a wide size range of capsular bags 28 such that second segments 112 of second ring 104 securely and substantially evenly engage inner wall 30 of the capsular bag 28 without the need for hinges 114.

Construction materials used to fabricate self-adjusting suspension system 100 include, but are not limited to, medically approved silicone elastomers, acrylic polymers, urethane polymers, combinations of these or other synthetic materials possessing good shape recovery characteristics.

In use, the self-adjusting suspension system 100 of the present invention is configured to reduce risk factors associated with lens replacement surgery. Typically, in preparation for surgical installation, an intraocular suspension system, or a composite intraocular lens/suspension system, is folded and/or compressed to fit into a cylindrical loading chamber that is integrated within the pipe channel of a syringe-style lens injector. Following this preliminary loading stage, the suspension system is compacted as it is pushed along the pipe channel to eventually exit for intraocular delivery through a dispensing port that is positioned within a vacant lens capsular bag 28 of a recipient eye 10.

The suspension system 100 of the present invention is optimally configured for surgical installation as described above. A consequence of the folding, compressing and/or compacting process required for syringe-style in vivo injection is that kinetic energy becomes stored within the structural elements of suspension system 100, namely first ring 102, second ring 104 and connectors 106. The moment that suspension system 100 exits the dispensing port, the stored kinetic energy is liberated, allowing the structural elements of the suspension system, such as hinges 114, spokes 124 or other tangential and/or radial biasing elements, to systematically revert back to a lower energy configuration.

When suspension system 100 is injected into the confines of a lens capsular bag 28 as described above, its full recovery back to a habitual, low energy configuration is restricted by wall 30 of capsular bag 28. In some embodiments opposing radial forces exerted by the resiliency of spokes 124 increase the separation between the first and second rings 102, 104 until second ring 104 becomes fixed upon inner wall 30 of a recipient lens capsular bag 28, for example at the equatorial perimeter or sulcus. Coincident with this radial expansion, opposing tangential forces exerted by the biasing element(s) within the structural elements of system 100, such as hinges 114, are liberated, resulting in an increase in the circumferential length of second ring 104 as it proceeds to conform to the entire length of the sulcus of a recipient lens capsular bag 28. These events mark the arrival of a new equilibrium of the driving forces that define the re-configured shape of a vacant lens capsular bag, 28 whereby second ring 104 is pressed taut against the equatorial perimeter region of the recipient lens capsule inner wall 30 and the first ring 102 is secured in a coaxial orientation upon the visual axis 31 of a recipient eye 10, throughout a pre-determined range of lens capsular bag sizes.

Accordingly, the self-adjusting suspension system 100 of the present invention, or a composite synthetic lens 18A/lens suspension system 100, is configured to unfold as a cohesive unit during surgical installation, reliably centering upon the visual axis 31 of a recipient eye 10 and reducing risks typically associated with lens replacement procedures, such as damage to or improper positioning of the lens 18A. In some embodiments suspension system 100, or a composite synthetic lens 18A/lens suspension system 100, expands in a manner visually reminiscent of an unfolding lotus flower from a compact, compressed state to an expanded, unfolded state by the simultaneous application of both radial and tangential forces as described herein. This manner of unfolding enables system 100 to self-adjust to the size and shape of the deployment site, such as a particular lens capsular bag 28 in vivo, without the need for significant surgical manipulation or other post-injection intervention.

As discussed above, in some embodiments connectors 106 may function as biasing elements. With reference to FIG. 8C, the joinder of each connector 106 to first ring 102 and/or second ring 104 may define a generally V-shaped compressible spring 200 having an apex or narrowed end 202 and ends 204. Apex 202 is spaced-apart from the plane of a portion of the first ring 102 supporting synthetic lens 18A. When suspension system 100 is deployed within the cavity of capsular bag 28 in the expanded configuration shown in FIG. 2, synthetic lens 18A may be generally aligned with a plane of the sulcus extending at the equatorial perimeter of inner wall 30. Apex 202 may be located in a plane posterior to the sulcus of capsular bag 28, as shown in FIG. 2. In other embodiments, apex 202 may be located anteriorly to the sulcus of the lens capsular bag 28, i.e. in a position further away from retina 20. As indicated above, the manner of unfolding enables system 100 to self-adjust or “auto-adapt” to the size, shape and orientation of the deployment site, such as a particular lens capsular bag 28 in vivo,

In some embodiments the separate components of self-adjusting suspension system 100 may be installed separately. For example, second ring 104 could be inserted into a recipient lens capsular bag 28 as a preliminary first step, unfolding and expanding to press itself taut along the inner wall 30 of the lens capsular bag 28, for example the equatorial perimeter or sulcus. A synthetic optical lens 18A/first ring 102 assembly with spoke-like projections 124 or other connectors 106 could then be placed as a secondary unit into the lens capsular bag 28 to fit into a predetermined space, or docking station, within the structures of the pre-installed second ring 104.

In summary, various embodiments of the invention described herein operate in a similar manner, i.e. opening in an aligned coaxial orientation upon the visual axis 31 of a recipient eye 10 with minimal potential complications when suspension system 100 is allowed to self-adjust between compressed and expanded configurations.

### Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Words that indicate directions such as “vertical”, “transverse”, “horizontal”, “upward”, “downward”, “forward”, “backward”, “inward”, “outward”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

For example, while processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed at different times

In addition, while elements are at times shown as being performed sequentially, they may instead be performed simultaneously or in different sequences. It is therefore intended that the following claims are interpreted to include all such variations as are within their intended scope.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

Various features are described herein as being present in “some embodiments”. Such features are not mandatory and may not be present in all embodiments. Embodiments of the invention may include zero, any one or any combination of two or more of such features. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible).

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

1. A lens suspension system positionable within an ocular capsular cavity comprising:
(a) a first ring configured for holding a lens within the ocular capsular cavity in a first plane;
(b) an expandable second ring at least partially surrounding the first ring, wherein the second ring comprises a plurality of spaced-apart second segments; and
(c) connectors for coupling the second ring to the first ring,
wherein the system is adjustable between a compressed configuration for deployment into the capsular cavity and an expanded configuration for engagement with an inner wall of the capsular cavity,
wherein the second ring is spaced a first distance apart from the first ring in the compressed configuration and wherein the second ring is spaced a second distance apart from the first ring in the expanded configuration, wherein the second distance is greater than the first distance,
wherein at least some of the connectors bias the system for adjustment from the compressed configuration to the expanded configuration, wherein the at least some of the connectors each comprise a first portion connected to one of the plurality of spaced-apart second segments and a second portion remote from first portion which extends in a second plane spaced-apart from the first plane in the expanded configuration,
and wherein the at least some of the connectors each form at least part of a respective compressible spring, wherein the second portion forms an apex of the spring.

2. The system as defined in claim 1, where the compressible spring is generally V-shaped in cross section.

3. The system as defined in claim 1, wherein, when the system is positioned within the ocular capsular cavity in the expanded configuration, the first plane is generally aligned with a plane of an equatorial perimeter of the inner wall and the second plane extends at a position either posterior or anterior of the plane of the equatorial perimeter.

4. The lens suspension system of claim 1, wherein the compressible spring acts to press a portion of the second ring against the inner wall when the system is positioned within the ocular capsular cavity in the expanded configuration.

5-28. (canceled)

29. The system as defined in claim 1, wherein adjacent pairs of the second segments are disconnected, each of the second segments being independently connected to the first ring.

30. A method of deploying an intraocular lens in an ocular capsular bag cavity having an internal wall comprising:
(a) providing a lens suspension system comprising a first ring configured for holding the lens, an expandable second ring at least partially surrounding the first ring, and connectors for coupling the second ring to the first ring; and
(b) introducing the lens suspension system into the cavity in a compressed configuration and allowing the system to adjust from the compressed configuration to an expanded configuration wherein outer surfaces of the second ring securely engage the internal wall of the cavity.

31-33. (canceled)

34. The method as defined in claim 30, wherein outer surfaces of the second ring are configured to securely engage an equatorial perimeter region or sulcus of the inner wall of the ocular capsular cavity when the system is deployed within the cavity and adjusted to the expanded configuration.

35-56. (canceled)

57. A method of deploying an intraocular lens in an ocular capsular bag cavity having an internal wall comprising:
(a) providing a lens suspension system comprising a first ring configured for holding the lens and an expandable second ring connectable to the first ring;
(b) introducing the second ring into the cavity in a compressed configuration and allowing the second ring to adjust from the compressed configuration to an expanded configuration wherein outer surfaces of the second ring securely engage the internal wall of the cavity;
(c) introducing the first ring into the cavity; and
(d) coupling the first ring to the second ring such that the second ring supports the first ring at an intraocular location maintaining the lens in alignment with a preferred visual axis of a recipient eye.

58-59. (canceled)

60. A method of deploying an intraocular lens in an ocular capsular bag cavity having an internal wall comprising:
(a) providing a lens suspension system as defined in claim 1,
(b) introducing the lens suspension system into the cavity in a compressed configuration and allowing the system to adjust from the compressed configuration to an expanded configuration wherein outer surfaces of the second ring securely engage the internal wall of the cavity.

61. The method as defined in claim 60 wherein the outer surfaces of the second ring engage an equatorial region of the capsular bag.

62. The method as defined in claim 60, wherein the suspension system is in a folded arrangement in the compressed configuration whereby a first portion of the system extends in a plane generally co-planar with a second portion of the system in the folded arrangement, whereby suspension system unfolds and expands after it is introduced into the cavity.

63-65. (canceled)

66. A lens suspension system positionable within an ocular capsular cavity comprising:
(a) a first ring configured for holding a lens; and
(b) an expandable second ring configured for at least partially surrounding the first ring, wherein the first ring is connectable to the second ring and wherein the system is adjustable between a compressed configuration for deployment into the capsular cavity and an expanded configuration for engagement with an inner wall of the capsular cavity wherein the first ring is connectable to the second ring by a plurality of compressible springs, each of the springs defining a trough between the first ring and the second ring, wherein a base of the trough extends either anteriorly or posteriorly of the lens when the system is deployed in the capsular cavity in the expanded configuration.

67. The lens suspension system of claim 66, wherein the second ring comprises a plurality of spaced-apart second segments and wherein each of the second segments is connected to the first ring by at least one resiliently deformable connector forming at least part of one of the compressible springs.

68. The lens suspension system of claim 67, wherein each of the compressible springs are generally V-shaped.

69. The lens suspension system of claim 67, wherein the first ring is resiliently deformable.

70. The lens suspension system of any one of claim 67, wherein the plurality of second segments are disconnected, wherein the second ring is interrupted at spaced intervals.

71. A lens suspension system positionable within an ocular capsular cavity comprising:
(a) a first ring configured for holding a lens;
(b) an expandable second ring at least partially surrounding the first ring; and
(c) a plurality of connectors for coupling the second ring to the first ring, wherein at least some of the connectors each form at least part of a respective compressible spring which is generally V-shaped in cross section, the spring having first and second portions joined at an apex,
wherein the system is adjustable between a compressed configuration for deployment into the capsular cavity and an expanded configuration for engagement with an inner wall of the capsular cavity.

72. The lens suspension system of claim 71, wherein a portion of the first ring supporting the lens extends in a first plane in the expanded configuration and wherein the apex is located in a second plane spaced-apart from the first plane in the expanded configuration.

73. The lens suspension system of claim 72, wherein, when the system is positioned within the ocular capsular cavity in the expanded configuration, the first plane is generally aligned with a plane of an equatorial perimeter of the inner wall and the second plane extends at a position either posterior or anterior of the plane of the equatorial perimeter.

74. The lens suspension system of claim 72, wherein the compressible spring acts to press a portion of the second ring against the inner wall when the system is positioned within the ocular capsular cavity in the expanded configuration.

75. A lens suspension system positionable within an ocular capsular cavity comprising:
(a) a first ring configured for holding a lens within the ocular capsular cavity;
(b) an expandable second ring at least partially surrounding the first ring, wherein the second ring comprises a plurality of spaced-apart second segments; and
(c) connectors for coupling the second segments to the first ring,
wherein the system is adjustable between a compressed configuration for deployment into the capsular cavity and an expanded configuration for engagement with an inner wall of the capsular cavity,
wherein the second ring is spaced a first distance apart from the first ring in the compressed configuration and wherein the second ring is spaced a second distance apart from the first ring in the expanded configuration, wherein the second distance is greater than the first distance,
wherein at least some of the connectors bias the system for adjustment from the compressed configuration to the expanded configuration,
wherein the at least some of the connectors each comprise a portion of a respective compressible spring defining a trough between the first ring and a respective one of the second segments, wherein a base of the trough extends either anteriorly or posteriorly of the lens when the system is deployed in the capsular cavity in the expanded configuration.

76. The system as defined in claim 75, wherein the at least some of the connectors comprises a first arm connecting the base of the trough to the first ring and a second arm connecting the base of the trough to the respective one of the second segments.

77. The system as defined in claim 76, wherein the first arm and second arm are resiliently deformable.

78. The system as defined in claim 75, wherein the compressible spring is generally V-shaped in cross section.

79. The system as defined in claim 75, wherein the base of the trough forms a narrowed end or apex of the compressible spring.

80. The system as defined in claim 75, wherein, when the system is positioned within the ocular capsular cavity in the expanded configuration, the lens extends in a first plane is generally aligned with a plane of an equatorial perimeter of the inner wall and wherein the base of the trough of each compressible spring extends in a second plane either posterior or anterior of the plane of the equatorial perimeter.

81. The system as defined in claim 75, wherein the compressible spring acts to press a portion of the second ring against the inner wall when the system is positioned within the ocular capsular cavity in the expanded configuration.

82. The system as defined in claim 75, wherein adjacent pairs of the second segments are disconnected, each of the second segments being independently connected to the first ring.
